(19) United States
(12) Patent Application Publication
(10) Pub. No.: US 2008/0144913 A1
(43) Pub. Date: Jun. 19, 2008

(54) COMPOSITE APPARATUS FOR RADIATION THERAPY AND ALIGNMENT CORRECTION DATA PRODUCING METHOD

(76) Inventor: Takayuki Yoshida, Tokyo (JP)

Correspondence Address: Patrick W. Rasche;Armstrong Teasdale LLP, Suite 2600, One Metropolitan Square, St. Louis, MO, 63102, US

(21) Appl. No.: 11/958,179

(22) Filed: Dec. 17, 2007

(30) Foreign Application Priority Data
Dec. 19, 2006 (JP) 2006-340663

(51) Int. Cl. G06K 9/00; A61B 6/00

(52) U.S. Cl. 382/132; 378/20

(57) ABSTRACT

Discrepancies in alignment between an X-ray CT apparatus and an apparatus for radiation therapy are to be corrected. A phantom for alignment correction (P) is imaged, and data for alignment correction for conversion of data (x, y, z) of the coordinates of the X-ray CT apparatus (100) into data (X, Y, Z) of the coordinates of the apparatus for radiation therapy (200) are produced. Then, the data in the coordinates of the X-ray CT apparatus (100) obtained by imaging the subject with the X-ray CT apparatus (100) are converted into data in the coordinates of the apparatus for radiation therapy (200).

### CROSS REFERENCE TO RELATED APPLICATIONS

This application claims the benefit of Japanese Patent Application No. 2006-340663 filed Dec. 19, 2006.

### BACKGROUND OF THE INVENTION

The field of the present invention relates to a composite apparatus for radiation therapy and an alignment correction data producing method, and more in detail to a composite apparatus for radiation therapy and an alignment correction data producing method capable of correcting discrepancies in alignment between an X-ray CT apparatus and an apparatus for radiation therapy.

A composite apparatus for radiation therapy comprising an X-ray CT apparatus, an apparatus for radiation therapy and a common table device shared thereby has already been known (see Patent JP-A No. 255160/2004 for instance).

### SUMMARY OF THE INVENTION

In the composite apparatus for radiation therapy, in order to locate a diseased part with its X-ray CT apparatus and cause its apparatus for radiation therapy to irradiate that position with X-rays, it was necessary to achieve precise alignment between the X-ray CT apparatus and the apparatus for radiation therapy.

However, there was a problem that the adjustment for this alignment took an extremely long time. There was another problem that, if there was misalignment, diagnosis or treatment had to be interrupted for a very long time for readjustment.

Therefore, an object of the present invention is to provide a composite apparatus for radiation therapy and an alignment correction data producing method capable of correcting discrepancies in alignment between an X-ray CT apparatus and an apparatus for radiation therapy.

The invention, according to its first aspect, provides a composite apparatus for radiation therapy comprising an X-ray CT apparatus, an apparatus for radiation therapy, and a table device common to the X-ray CT apparatus and the apparatus for radiation therapy, wherein the X-ray CT apparatus is provided with data for alignment correction for converting data in the coordinates of the X-ray CT apparatus into data in the coordinates of the apparatus for radiation therapy and correcting device for converting data in the coordinates of the X-ray CT apparatus obtained by imaging into data in the coordinates of the apparatus for radiation therapy on the basis of the data for alignment correction.

As this composite apparatus for radiation therapy according to the first aspect converts data in the coordinates of the X-ray CT apparatus obtained by imaging into data in the coordinates of the apparatus for radiation therapy on the basis of the data for alignment correction, discrepancies, if any, in alignment between the X-ray CT apparatus and the apparatus for radiation therapy can be corrected. Also, since discrepancies in alignment can be permitted, the time taken to adjust the alignment between the X-ray CT apparatus and the apparatus for radiation therapy can be reduced.

According to the second aspect of the invention, there is provided a composite apparatus for radiation therapy according to the first aspect, wherein the X-ray CT apparatus is equipped with display device for displaying an image on the basis of the data in the coordinates of the apparatus for radiation therapy.

As the composite apparatus for radiation therapy according to this second aspect displays on the X-ray CT apparatus an image having gone through correction of discrepancies in alignment between the X-ray CT apparatus and the apparatus for radiation therapy, the diseased part to be irradiated by the apparatus for radiation therapy with its radiation can be accurately located on the X-ray CT apparatus.

According to the third aspect of the invention, there is provided a composite apparatus for radiation therapy according to the first or second aspect, wherein the top plate of the table device can rotate within a horizontal plane.

In the composite apparatus for radiation therapy according to the third aspect, the object of imaging can be shifted between the X-ray CT apparatus and the apparatus for radiation therapy by rotating the top plate of the table device within a horizontal plane.

According to the fourth aspect of the invention, there is provided a composite apparatus for radiation therapy according to any of the first through third aspect, wherein the gantry of the X-ray CT apparatus can horizontally shift.

In the composite apparatus for radiation therapy according to the fourth aspect, when the object of imaging is to be shifted between the X-ray CT apparatus and the apparatus for radiation therapy, the X-ray CT apparatus can be set aside by horizontally shifting the gantry of the X-ray CT apparatus.

According to the fifth aspect of the invention, there is provided a composite apparatus for radiation therapy according to any of the first through fourth aspects, wherein the data for alignment correction is produced based on data on a phantom for alignment correction in coordinates of the apparatus for radiation therapy and data on the phantom for alignment correction in coordinates of the X-ray CT apparatus, wherein said data on the phantom for alignment correction in coordinates of the apparatus for radiation therapy is determined by positioning said phantom on the table device in the coordinates system of the apparatus for radiation therapy, and said data on the phantom for alignment correction of in coordinates of the X-ray CT apparatus is obtained by moving said phantom placed on the table device relative to the X-ray CT apparatus and imaging said phantom.

In the composite apparatus for radiation therapy according to the fifth aspect, data for alignment correction for converting data in the coordinates of the X-ray CT apparatus into data in the coordinates of the apparatus for radiation therapy can be appropriately produced by using a phantom for alignment correction.

According to the sixth aspect of the invention, there is provided a composite apparatus for radiation therapy according to the fifth aspect, wherein the data for alignment correction are produced by using four or more characteristic points which are not on the same plane of the phantom for alignment correction.

In the composite apparatus for radiation therapy according to the sixth aspect, data for alignment correction which permit correction of three-dimensional discrepancies can be produced by using four or more characteristic points which are not on the same plane of the phantom for alignment correction.

According to the seventh aspect of the invention, there is provided a composite apparatus for radiation therapy according to the fifth or sixth aspect, wherein the phantom for alignment correction is a three-dimensional frame body formed of tungsten wires.

In the composite apparatus for radiation therapy according to the seventh aspect, the phantom for alignment correction can be reduced in weight.

According to the eighth aspect of the invention, there is provided a composite apparatus for radiation therapy according to the seventh aspect, wherein the data for alignment correction are produced by comparing coordinates with reference to grid points where the tungsten wires cross each other as characteristic points.

In the composite apparatus for radiation therapy according to the eighth aspect, coordinates are easier to compare because grid points where the tungsten wires cross each other are used as characteristic points.

According to the ninth aspect of the invention, there is provided a composite apparatus for radiation therapy according to any of the first through eighth aspects, wherein the data for alignment correction are data for affine transformation.

In the composite apparatus for radiation therapy according to the ninth aspect, data in the coordinates of the X-ray CT apparatus can be converted into data in the coordinates of the apparatus for radiation therapy by affine transformation.

According to the tenth aspect of the invention, there is provided an alignment correction data producing method comprising the steps of: positioning a phantom for alignment correction placed on the table device of a composite apparatus for radiation therapy which comprises an X-ray CT apparatus, an apparatus for radiation therapy, and the table device common to the X-ray CT apparatus and the apparatus for radiation therapy, by the coordinates of the apparatus for radiation therapy; acquiring data on the phantom for alignment correction by the coordinates of the X-ray CT apparatus by moving relative to the X-ray CT apparatus the phantom for alignment correction remaining placed on the table device to image the phantom; and producing data for alignment correction for converting data in the coordinates of the X-ray CT apparatus into data in the coordinates of the apparatus for radiation therapy, on the basis of data on the phantom for alignment correction in the coordinates of the apparatus for radiation therapy determined by the positioning and said data on the phantom for alignment correction in the coordinates of the X-ray CT apparatus.

By the alignment correction data producing method according to the tenth aspect, data for alignment correction for converting data in the coordinates of X-ray CT apparatus into data in the coordinates of the apparatus for radiation therapy can be appropriately produced by using a phantom for alignment correction.

According to the eleventh aspect of the invention, there is provided an alignment correction data producing method according to the tenth aspect, wherein the relative moment is accomplished by the rotation of the top plate of the table device within a horizontal plane and the horizontal shift of the gantry of the X-ray CT apparatus.

By the alignment correction data producing method according to the eleventh aspect, the object of imaging can be moved between the X-ray CT apparatus and the apparatus for radiation therapy by horizontally shifting the gantry of the X-ray CT apparatus and keeping it set aside and rotating the top plate of the table device. Incidentally, when the object of imaging is to be moved from the apparatus for radiation therapy to the X-ray CT apparatus, the gantry of the X-ray CT apparatus is horizontally shifted to return from the set-aside position to the imaging position after rotating the top plate of the table device within the horizontal plane.

According to the twelfth aspect of the invention, there is provided an alignment correction data producing method according to the tenth or eleventh aspect, wherein the data for alignment correction are produced by using four or more characteristic points which are not on the same plane of the phantom for alignment correction.

By the alignment correction data producing method according to the twelfth aspect, data for alignment correction which permit correction of three-dimensional discrepancies can be produced by using four or more characteristic points which are not on the same plane of the phantom for alignment correction.

According to the thirteenth aspect of the invention, there is provided an alignment correction data producing method according to any of the tenth through twelfth aspects, wherein a three-dimensional frame body formed of tungsten wires is used as the phantom for alignment correction.

By the alignment correction data producing method according to the thirteenth aspect, the phantom for alignment correction can be reduced in weight.

According to the fourteenth aspect of the invention, there is provided an alignment correction data producing method according to the thirteenth aspect, wherein each side of the three-dimensional frame body measures 30 cm or more.

By the alignment correction data producing method according to the fourteenth aspect, sufficient accuracy of discrepancy detection can achieved because it uses a three-dimensional frame body each side of which measures 30 cm or more.

According to the fifteenth aspect of the invention, there is provided an alignment correction data producing method according to the thirteenth or fourteenth aspect, wherein the data for alignment correction are produced by comparing coordinates with reference to grid points where the tungsten wires cross each other as characteristic points.

By the alignment correction data producing method according to the fifteenth aspect, coordinates are easier to compare because grid points where the tungsten wires cross each other are used as characteristic points.

According to the sixteenth aspect of the invention, there is provided an alignment correction data producing method according to any of the tenth through fifteenth aspects, wherein the data for alignment correction are data for affine transformation.

By the alignment correction data producing method according to the sixteenth aspect, data in the coordinates of the X-ray CT apparatus can be converted into data in the coordinates of the apparatus for radiation therapy by affine transformation.

The composite apparatus for radiation therapy and the alignment correction data producing method according to the invention can correct discrepancies in alignment, if any, between the X-ray CT apparatus and the apparatus for radiation therapy. It can also reduce the time taken to adjust the alignment between the X-ray CT apparatus and the apparatus for radiation therapy.

### BRIEF DESCRIPTION OF THE DRAWINGS

FIG. 1 is a block diagram of the X-ray CT apparatus included in the composite apparatus for radiation therapy pertaining to Embodiment 1.

FIG. 2 is a profile showing the essential part of the composite apparatus for radiation therapy pertaining to Embodiment 1.

FIG. 3 is a perspective view showing one example of phantom for alignment correction.

FIG. 4 is a flow chart showing the procedure of producing data for alignment correction pertaining to Embodiment 1.

FIG. 5 is a profile showing a shift of a phantom for alignment correction to the X-ray CT apparatus.

FIG. 6 is a profile showing the pickup of a phantom for alignment correction by the X-ray CT apparatus.

7(a) and 7(b) are conceptual diagrams showing the coordinates of X imaging data of the phantom for alignment correction by the X-ray CT apparatus and the coordinates of the phantom for alignment correction the coordinates in the apparatus for radiation therapy.

FIG. 8 is a flow chart showing the procedure of subject imaging pertaining to Embodiment 1.

### DETAILED DESCRIPTION OF THE INVENTION

The present invention will be described in further detail below with reference to the mode for carrying it out shown in the drawings. This, however, is nothing to limit the invention.

### Embodiment 1

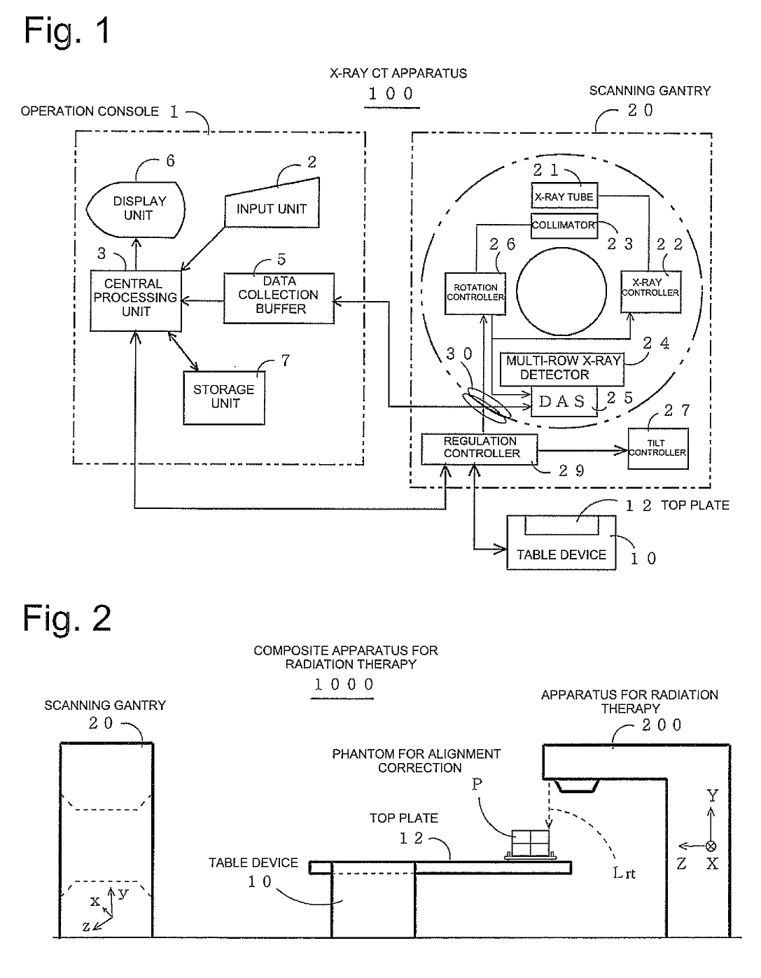

FIG. 1 is a configurational block diagram of an X-ray CT apparatus 100 included in a composite apparatus for radiation therapy (denoted by 1000 in FIG. 2) pertaining to Embodiment 1. This X-ray CT apparatus 100 is equipped with an operation console 1, a table device 10 and a scanning gantry 20.

The operation console 1 is provided with an input unit 2 for accepting inputs by the operator, a central processing unit 3 for executing data for alignment correction and subject imaging, a data collection buffer 5 for collecting projection data acquired by the scanning gantry 20, a display unit 6 for displaying a tomogram reconstructed from the projection data, and a storage unit 7 for storing programs, data including data for alignment correction and X-ray tomograms.

The table device 10 has a top plate 12 which, mounted with a subject, moves between inside and outside the bore (hollow part) of the scanning gantry. The top plate 12 can rotate within a horizontal plane, linearly shift horizontally, and ascend and descend.

The scanning gantry 20 is equipped with an X-ray tube 21, an X-ray controller 22, a collimator 23, a multi-row X-ray detector 24, a DAS (Data Acquisition System) 25, a rotation controller 26 for turning the X-ray tube 21 and the like around the body axis of the subject, a tilt controller 27 for controlling the scanning gantry 20 when it is to be inclined before or behind the rotation axis, a regulation controller 29 which exchanges control signals and the like with the operation console 1 and the bed device 10, and a slip ring 30.

Projection data obtained by the multi-row X-ray detector 24 undergo A/D conversion by the DAS 25 and are transferred to the data collection buffer 5 via the slip ring 30.

The central processing unit 3 subjects the projection data collected into the data collection buffer 5 to various pretreatments including correction and to image reconstruction to produce a tomogram, and displays the tomogram on the display unit 6.

FIG. 2 is a profile showing the essential part of the composite apparatus for radiation therapy 1000 pertaining to Embodiment 1.

The composite apparatus for radiation therapy 1000 comprises the X-ray CT apparatus 100 of FIG. 1 (please note that only scanning gantry 20 and table device 10 of X-ray CT apparatus are displayed in FIG. 2) and an apparatus for radiation therapy 200 and the common table device 10. FIG. 2 also illustrates a phantom for alignment correction P positioned on table device 10 and a laser pointer Lrt.

The coordinates of the X-ray CT apparatus 100 is represented by (x, y, z), and the coordinates of the apparatus for radiation therapy 200, by (X, Y, z).

When the object of treatment is to be moved from the scanning gantry 20 of the X-ray CT apparatus 100 to the apparatus for radiation therapy 200, the top plate 12 of the table device 10 is turned within the horizontal plane after setting aside the scanning gantry 20 by horizontal shifting.

When the object of imaging is to be moved from the apparatus for radiation therapy 200 to the scanning gantry 20 of the X-ray CT apparatus 100, after the top plate 12 of the table device 10 is turned within the horizontal plane in a state in which the scanning gantry 20 is kept in a set-aside position, the scanning gantry 20 of the X-ray CT apparatus 100 is returned from the set-aside position to the imaging position by horizontal shifting.

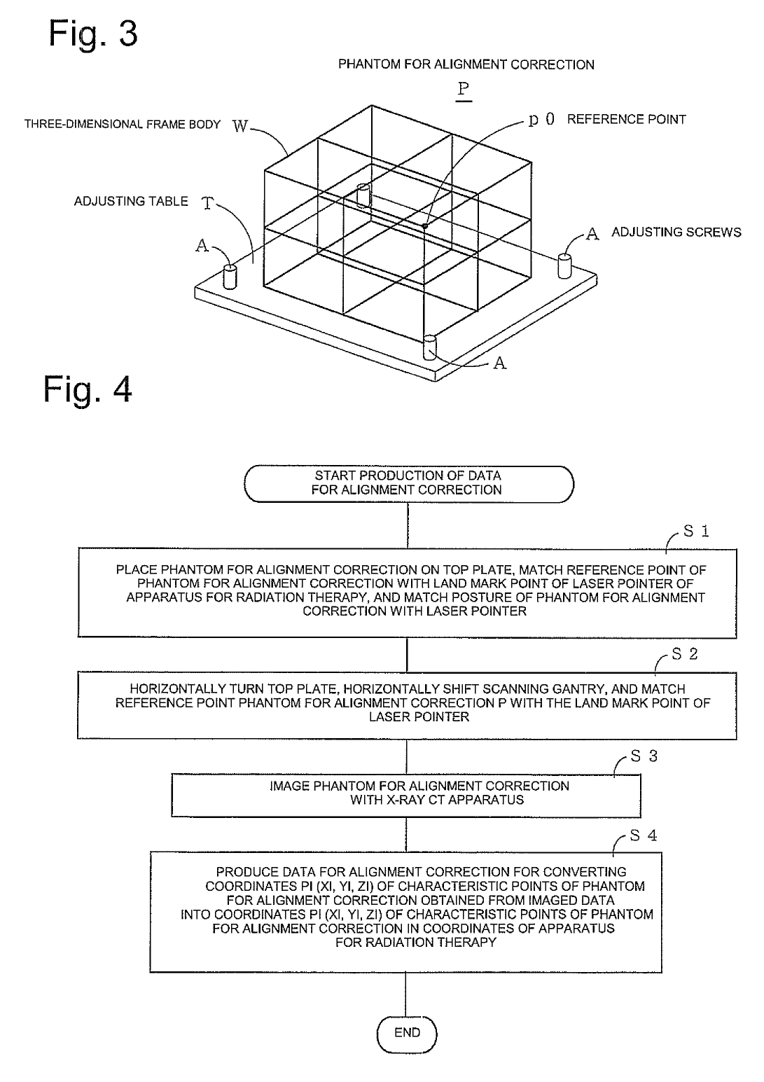

Referring to FIG. 3, the phantom for alignment correction P comprises a three-dimensional frame body W formed of tungsten wires and an adjusting table T on which the three-dimensional frame body W is to be mounted.

The three-dimensional frame body W is a rectangular prism each side of which is 30 cm or longer.

The posture of the three-dimensional frame body W can be adjusted with adjusting screws A of the adjusting table T.

Grid points where the tungsten wires of the three-dimensional frame body W cross each other constitute characteristic points and one of the characteristic points constitutes a reference point p0.

FIG. 4 is a flow chart showing the procedure of producing data for alignment correction using the composite apparatus for radiation therapy 1000.

At step S1, as shown in FIG. 2, the phantom for alignment correction P is placed on the top plate 12, the reference point p0 of the phantom for alignment correction P is matched with the land mark point of the laser pointer Lrt of the apparatus for radiation therapy 200, and at the same time the posture of the phantom for alignment correction P is adjusted according to the laser pointer Lrt. As the geometrical structure of the phantom for alignment correction P is known, the coordinates pi (Xi, Yi, Zi) of each of the characteristic points of the phantom for alignment correction P in the coordinates of the apparatus for radiation therapy 200 are determined by this adjustment.

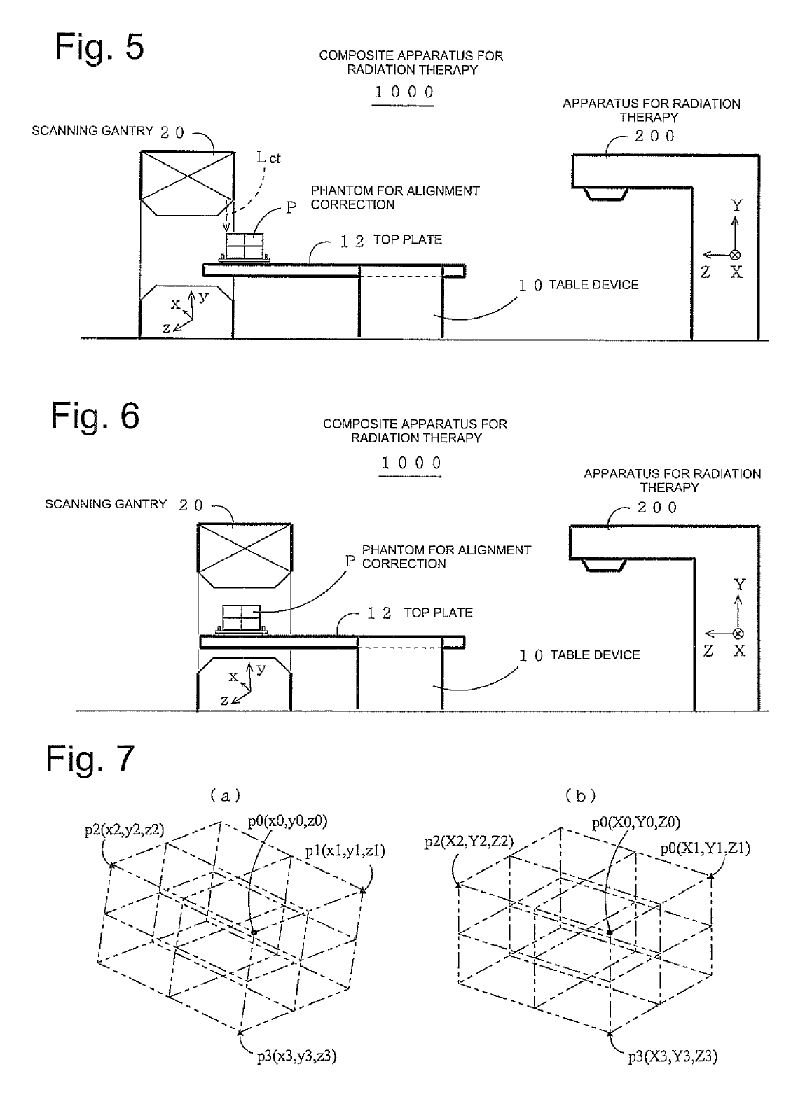

At step S2, as shown in FIG. 5, the top plate 12 is horizontally turned, the scanning gantry 20 is horizontally shifted, and the reference point p0 of the phantom for alignment correction P is matched with the land mark point of the laser pointer Lct of the X-ray CT apparatus 200.

At step S3, as shown in FIG. 6, the phantom for alignment correction P is imaged with the apparatus for radiation therapy 200, and the coordinates pi (xi, yi, zi) of each characteristic point of the phantom for alignment correction P in the coordinates of X-ray CT apparatus 100 are obtained.

At step S4, data for alignment correction for converting the coordinates pi (xi, yi, zi) of each characteristic point of the phantom for alignment correction P in the coordinates of the X-ray CT apparatus 100 shown in FIG. 7(a) into the coordinates pi (Xi, Yi, Zi) of each characteristic point of the phantom for alignment correction P in the coordinates of the apparatus for radiation therapy 200 shown in FIG. 7(b) are produced. The data for alignment correction are, for instance, data for affine transformation. The processing is ended then.

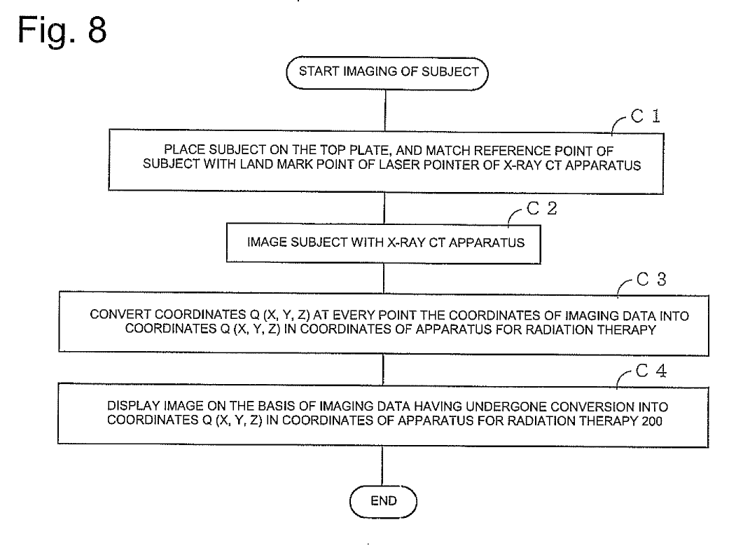

FIG. 8 is a flow chart showing the procedure of subject imaging using the X-ray CT apparatus 100.

At step C1, the subject is placed on the top plate 12, and the reference point of the subject is matched with the land mark point of the laser pointer Lct of the X-ray CT apparatus 100.

At step C2, the subject is imaged with the X-ray CT apparatus 100 to obtain imaging data.

At step C3, the coordinates q (x, y, z) in the coordinates of the X-ray CT apparatus 100 at every point of the imaging data are converted with data for alignment correction into the coordinates q (X, Y, Z) in the coordinates of the apparatus for radiation therapy 200.

At step C4, on the basis of the imaging data having undergone conversion into the coordinates q (X, Y, Z) in the coordinates of the apparatus for radiation therapy 200, a tomogram is displayed. The processing is ended then.

By using the composite apparatus for radiation therapy 1000 of Embodiment 1, discrepancies in alignment, if any, between the X-ray CT apparatus 100 and the apparatus for radiation therapy 200 can be corrected. Also, the time taken to adjust the alignment between the X-ray CT apparatus 100 and the apparatus for radiation therapy 200 can be reduced.

The composite apparatus for radiation therapy and the alignment correction data producing method according to the present invention can be utilized for accurately locating a diseased part to be treated by radiation therapy.

1. A composite apparatus for radiation therapy comprising an X-ray CT apparatus, an apparatus for radiation therapy, and a table device common to the X-ray CT apparatus and the apparatus for radiation therapy,
wherein the X-ray CT apparatus includes: data for alignment correction for converting data in the coordinates of the X-ray CT apparatus into data in the coordinates of the apparatus for radiation therapy; and a correcting device for converting data in the coordinates of the X-ray CT apparatus obtained by imaging into data in the coordinates of the apparatus for radiation therapy on the basis of the data for alignment correction.

2. The composite apparatus for radiation therapy according to claim 1, wherein the X-ray CT apparatus is provided with a display device for displaying an image on the basis of the data converted to the data in the coordinates of the apparatus for radiation therapy.

3. The composite apparatus for radiation therapy according to claim 1, wherein the table device further comprises a top plate that can rotate within a horizontal plane.

4. The composite apparatus for radiation therapy according to claim 1, wherein the X-ray CT apparatus further comprises a gantry that can horizontally shift.

5. The composite apparatus for radiation therapy according to claim 3, wherein the X-ray CT apparatus further comprises a gantry that can horizontally shift.

6. The composite apparatus for radiation therapy according to claim 1, wherein the data for alignment correction is produced based on data on a phantom for alignment correction in coordinates of the apparatus for radiation therapy and data on the phantom for alignment correction in coordinates of the X-ray CT apparatus, wherein said data on the phantom for alignment correction in coordinates of the apparatus for radiation therapy is determined by positioning said phantom on the table device in the coordinates system of the apparatus for radiation therapy, and said data on the phantom for alignment correction of in coordinates of the X-ray CT apparatus is obtained by moving said phantom placed on the table device relative to the X-ray CT apparatus and imaging said phantom.

7. The composite apparatus for radiation therapy according to claim 6, wherein the data for alignment correction are produced by using four or more characteristic points which are not on the same plane of the phantom for alignment correction.

8. The composite apparatus for radiation therapy according to claim 6, wherein the phantom for alignment correction is a three-dimensional frame body formed of tungsten wires.

9. The composite apparatus for radiation therapy according to claim 8, wherein the data for alignment correction are produced by comparing coordinates with reference to grid points where the tungsten wires cross each other as characteristic points.

10. The composite apparatus for radiation therapy according to claim 1, wherein the data for alignment correction are data for affine transformation.

11. The composite apparatus for radiation therapy according to claim 6, wherein the data for alignment correction are data for affine transformation.

12. An alignment correction data producing method comprising the steps of:
positioning a phantom for alignment correction placed on the table device of a composite apparatus for radiation therapy which comprises an X-ray CT apparatus, an apparatus for radiation therapy, and the table device common to the X-ray CT apparatus and the apparatus for radiation therapy, in the coordinates of the apparatus for radiation therapy;
acquiring data on the phantom for alignment correction in the coordinates of the X-ray CT apparatus by moving relative to the X-ray CT apparatus the phantom for alignment correction placed on the table device to image the phantom; and
producing data for alignment correction for converting data in the coordinates of the X-ray CT apparatus into data in the coordinates of the apparatus for radiation therapy, on the basis of data on the phantom for alignment correction in the coordinates of the apparatus for radiation therapy determined by the positioning and said data on the phantom for alignment correction in the coordinates of the X-ray CT apparatus.

13. The alignment correction data producing method according to claim 12, wherein the relative moment is accomplished by the rotation of the top plate of the table device within a horizontal plane and the horizontal shift of the gantry of the X-ray CT apparatus.

14. The alignment correction data producing method according to claim 12, wherein the data for alignment correction are produced by using four or more characteristic points which are not on the same plane of the phantom for alignment correction.

15. The alignment correction data producing method according to claim 12, wherein a three-dimensional frame body formed of tungsten wires is used as the phantom for alignment correction.

16. The alignment correction data producing method according to claim 15, wherein each side of the three-dimensional frame body measures 30 cm or more.

17. The alignment correction data producing method according to claim 15, wherein the data for alignment correction are produced by comparing coordinates with reference to grid points where the tungsten wires cross each other as characteristic points.

18. The alignment correction data producing method according to claim 12, wherein the data for alignment correction are data for affine transformation.
